(19) United States
(12) Patent Application Publication
(10) Pub. No.: US 2026/0191887 A1
(43) Pub. Date: Jul. 9, 2026

(54) METHODS OF TREATING INFLAMMATION

(71) Applicant: THERAPEUTIKOS, INC., Harlingen, TX (US)

(72) Inventors: Ameer E. HASSAN, Harlingen, TX (US); Yousef Hasan Ahmad KHALILI, Dubai (AE)

(21) Appl. No.: 18/856,386

(22) PCT Filed: Apr. 20, 2023
(86) PCT No.: PCT/US2023/065990

§ 371 Date: Oct. 11, 2024

Related U.S. Application Data
(60) Provisional application No. 63/333,281, filed on Apr. 21, 2022.

(51) Int. Cl. A61K 31/7008; A61K 45/06; A61P 29/00

(52) U.S. Cl. CPC A61K 31/7008; A61K 45/06; A61P 29/00

(57) ABSTRACT

The present disclosure relates to anti-inflammatory pharmaceutical agents, and specifically relates to compounds, compositions and methods for treating inflammation and inflammation-associated disorders, such as arthritis.

### CROSS-REFERENCE TO RELATED APPLICATIONS

This application claims priority under 35 U.S.C. § 119(e) to U.S. Provisional Application Ser. No. 63/333,281 filed on Apr. 21, 2022, the entire disclosure of which is incorporated herein by reference.

### REFERENCE TO SEQUENCE LISTING SUBMITTED ELECTRONICALLY

Incorporated by reference in its entirety is a computer-readable nucleotide/amino acid sequence listing submitted concurrently herewith and identified as follows: as a 16 kilobytes xml file named “83864-388942_SL.xml”, created on Apr. 17, 2023.

### FIELD

The present disclosure relates to anti-inflammatory pharmaceutical agents, and specifically relates to compounds, compositions and methods for treating inflammation and inflammation-associated disorders, such as arthritis.

### BACKGROUND

Since its discovery as a biologically active molecule in the late 1980s, nitric oxide (NO) has been found to play an important role as signal molecule in many parts of the organism as well as cytotoxic or regulatory effector molecule of the innate immune response. NO, which is synthesized by nitric oxide synthase (NOS), is the smallest known bioactive molecule, and can be produced by a variety of cells. NO plays an important role in neurotransmission, vascular function, host defense, and immune regulation.

Three NOS isoforms have been identified: neuronal nitric oxide synthase (nNOS), inducible nitric oxide synthase (iNOS), and endothelial nitric oxide synthase (eNOS). The signal molecule NO is synthesized on demand for short periods of time (seconds to minutes) following enzyme activation of constitutively expressed endothelial NO synthase (eNOS) or neuronal NO synthase (nNOS). In contrast, the inducible NO synthase (iNOS) is expressed only after cell activation and then produces NO for comparatively long periods of time (hours to days). iNOS can be produced by a variety of cells after induction by cytokines or other stimuli. Thus, regulated short pulsative synthesis versus constant NO production differentiates between physiological and pathophysiological actions of NO.

NO is an important pro-inflammatory mediator with an effect on the immune system. Indeed, NO plays a dual role in the process of immunoinflammation. On the one hand, NO can kill microorganisms and has a protective effect on the body, helping to fight against various viruses, such as herpes simplex virus (HSV). On the other hand, NO can damage normal tissue cells to generate pathogenic effects, and is widely involved in the development of various diseases, such as Boma disease. According to existing research, macrophages and other effector cells, including neutrophils, monocytes, and endothelial cells, are the main effector cells involved in the antimicrobial effects of NO.

The iNOS gene is under the transcriptional control of a variety of inflammatory mediators such as cytokines, lipopolysaccharide (LPS), and others. Aberrant expression of iNOS plays a crucial role in many inflammatory diseases such as rheumatoid arthritis (RA), osteoarthritis (OA), colitis, asthma, multiple sclerosis, systemic sclerosis, Sjögren's syndrome, bronchiectasis, idiopathic pulmonary fibrosis, atherosclerotic plaques, ulcerative colitis, Crohn's disease, necrotizing enterocolitis, Coeliac disease, glomerulonephritis, dialated cardiomyopathy, cutaneous lupus erythematosus, systemic lupus erythematosus, dermatitis, periapical periodontitis, and psoriasis. For example, iNOS generated NO seems to be crucially involved in the pathomechanisms of OA, and it contributes to the OA pathogenesis by modulating ECM homeostasis and cytokines expression, causing oxidative damage and chondrocyte apoptosis. Using immunocytochemistry, reverse transcriptase-polymerase chain reaction (RT-PCR), and in situ hybridization, iNOS expression has been described in rheumatoid arthritis (RA), multiple sclerosis (MS), and Sjögren's syndrome.

Numerous studies suggest a similar role of iNOS activity in human disease. However, it has still to be shown whether inhibiting iNOS-derived NO in human patients will protect from tissue-destructive processes in OA, RA, MS type-1 diabetes, and the like. Accordingly, there is a significant unmet need for improved therapies for a variety of inflammatory diseases, including rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.

### SUMMARY

In one aspect, the disclosure provides a method of treating a subject having an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like, comprising administering a therapeutically effective amount of a compound of the formula

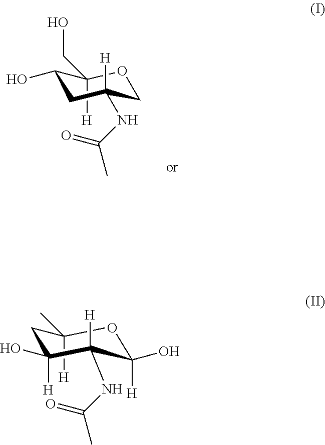

or a pharmaceutically acceptable salt thereof.

In another aspect, the disclosure provides a pharmaceutical composition comprising a compound of the formula

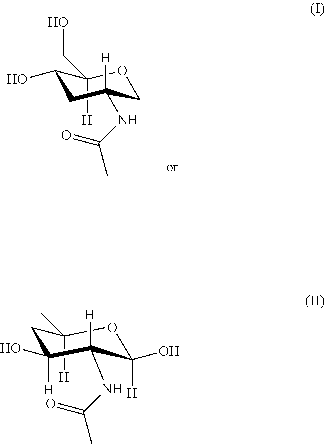

or a pharmaceutically acceptable salt thereof, and optionally a pharmaceutically acceptable carrier or excipient. In some embodiments, the compound in the pharmaceutical composition is in a therapeutically effective amount for treating an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.

In another aspect, the disclosure provides a use of a compound of the formula

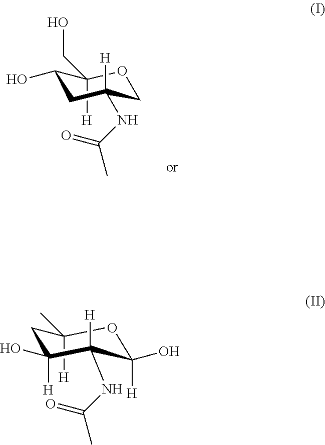

or a pharmaceutically acceptable salt thereof, in the preparation of a medicament for treating a subject having an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.

Additional embodiments, features, and advantages of the disclosure will be apparent from the following detailed description and through practice of the disclosure. The compounds, methods, and compositions of the present disclosure can be described as embodiments in any of the following enumerated clauses. It will be understood that any of the embodiments described herein can be used in connection with any other embodiments described herein to the extent that the embodiments do not contradict one another.
1. A method of treating a subject having an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like, comprising administering a therapeutically effective amount of a compound of the formula

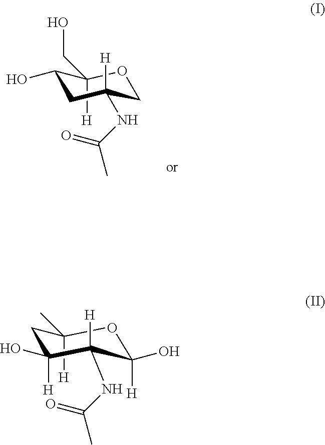

or a pharmaceutically acceptable salt thereof.
2. The method of clause 1, wherein the inflammatory disease is rheumatoid arthritis (RA).
3. The method of clause 1, wherein the inflammatory disease is osteoarthritis (OA).
4. The method of any one of clauses 1 to 3, wherein the therapeutically effective amount treats one or more symptoms of the inflammatory disease.
5. The method of any of one of clauses 1 to 4, wherein the compound is administered intravenously, orally, subcutaneously, buccally, transdermally, or nasally.
6. The method of any of one of clauses 1 to 5, wherein the compound is administered orally.
7. The method of any one of clauses 1 to 6, wherein the therapeutically effective amount of the inflammatory disease is in the range of about 1 mg to about 1000 mg.
8. The method of any one of clauses 1 to 7, wherein the therapeutically effective amount of the compound is administered once weekly, twice weekly, once a day (QD), twice a day (BID), or three times a day (TID).
9. The method of any one of clauses 1 to 8, wherein the therapeutically effective amount of the compound inhibits NO production in the patient.
10. The method of any one of clauses 1 to 9, wherein the therapeutically effective amount of the compound inhibits iNOS gene expression in the patient.
11. The method of any one of clauses 1 to 10, further comprising administering to the patient of one or more additional therapeutic agents.
12. The method of clause 10, wherein the one or more additional therapeutic agents is an a corticosteroid, a salicylate, an acetic acid derivative, an enolic acid (oxicam) derivative, a propionic acid derivative, an anthranilic acid derivative, or a Cox-2 inhibitor.
13. The method of clause 10, wherein the one or more additional therapeutic agents is selected from the group consisting of aspirin, diflunisal, salsalate, diclofenac, etodolac, indomethacin, ketorolac, nabumetone, sulindac, tolmetin, meloxicam, piroxicam, ibuprofen, naproxen, ketoprofen, fenoprofen, flurbiprofen, oxoprozin, mefenamic acid, celecoxib, cortisone, prednisone, and methylprednisone.
14. A pharmaceutical composition comprising a compound of the formula

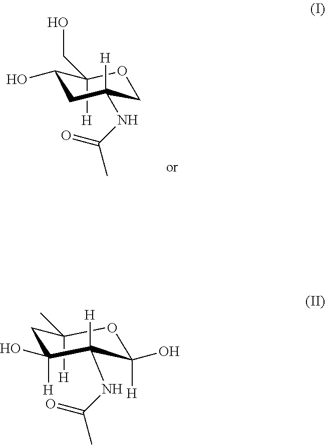

or a pharmaceutically acceptable salt thereof, and optionally a pharmaceutically acceptable carrier or excipient, wherein the compound is in a therapeutically effective amount for treating an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.
15. The pharmaceutical composition of clause 14, wherein the compound is in an amount of about 1 mg to about 1000 mg in the composition.
16. The pharmaceutical composition of clause 14 or 15, wherein the therapeutically effective amount of the compound inhibits NO production in the patient.
17. The pharmaceutical composition of any one of clauses 14 to 16, wherein the therapeutically effective amount of the compound inhibits iNOS gene expression in the patient.
18. The pharmaceutical composition of any one of clauses 14 to 17, wherein the composition is to be administered intravenously, orally, subcutaneously, buccally, transdermally, or nasally.
19. The pharmaceutical composition according to any one of clauses 14 to 18, wherein the composition is to be administered orally.
20. Use of a compound of the formula

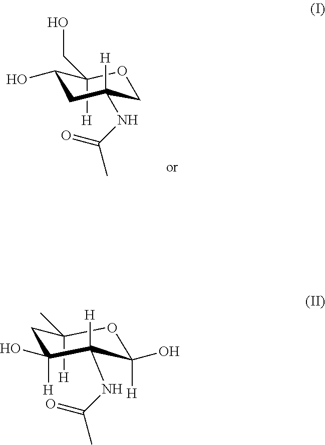

or a pharmaceutically acceptable salt thereof, in the preparation of a medicament for treating a subject having an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.
21. The use of clause 20, wherein the inflammatory disease is rheumatoid arthritis (RA).
22. The use of clause 20, wherein the inflammatory disease is osteoarthritis (OA).
23. The use of any one of clauses 20 to 22, wherein the therapeutically effective amount treats one or more symptoms of the inflammatory disease.
24. The use of any of one of clauses 20 to 23, wherein the compound is administered intravenously, orally, subcutaneously, buccally, transdermally, or nasally.
25. The use of any of one of clauses 20 to 24, wherein the compound is administered orally.
26. The use of any one of clauses 20 to 25, wherein the therapeutically effective amount of the inflammatory disease is in the range of about 1 mg to about 1000 mg.
27. The use of any one of clauses 20 to 26, wherein the therapeutically effective amount of the compound is administered once weekly, twice weekly, once a day (QD), twice a day (BID), or three times a day (TID).
28. The use of any one of clauses 20 to 27, wherein the therapeutically effective amount of the compound inhibits NO production in the patient.
29. The use of any one of clauses 20 to 28, wherein the therapeutically effective amount of the compound inhibits iNOS gene expression in the patient.
30. The use of any one of clauses 20 to 29, the treating further comprising administering to the patient of one or more additional therapeutic agents.
31. The use of clause 30, wherein the one or more additional therapeutic agents is an a corticosteroid, a salicylate, an acetic acid derivative, an enolic acid (oxicam) derivative, a propionic acid derivative, an anthranilic acid derivative, or a Cox-2 inhibitor.
32. The use of clause 30, wherein the one or more additional therapeutic agents is selected from the group consisting of aspirin, diflunisal, salsalate, diclofenac, etodolac, indomethacin, ketorolac, nabumetone, sulindac, tolmetin, meloxicam, piroxicam, ibuprofen, naproxen, ketoprofen, fenoprofen, flurbiprofen, oxoprozin, mefenamic acid, celecoxib, cortisone, prednisone, and methylprednisone.
33. A compound of the formula

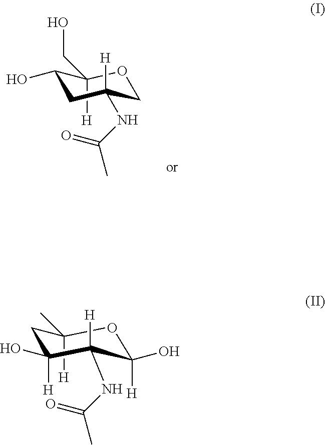

or a pharmaceutically acceptable salt thereof, for use in a method of treating an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like, in a subject.
34. The compound of clause 33, wherein the inflammatory disease is rheumatoid arthritis (RA).
35. The compound of clause 33, wherein the inflammatory disease is osteoarthritis (OA).
36. The compound of any one of clauses 33 to 35, wherein the therapeutically effective amount treats one or more symptoms of the inflammatory disease.
37. The compound of any of one of clauses 33 to 36, wherein the compound is administered intravenously, orally, subcutaneously, buccally, transdermally, or nasally.
38. The compound of any of one of clauses 33 to 37, wherein the compound is administered orally.
39. The compound of any one of clauses 33 to 38, wherein the therapeutically effective amount of the inflammatory disease is in the range of about 1 mg to about 1000 mg.
40. The compound of any one of clauses 33 to 39, wherein the method comprises administering a therapeutically effective amount of the compound once weekly, twice weekly, once a day (QD), twice a day (BID), or three times a day (TID).
41. The compound of any one of clauses 33 to 40, wherein the method comprises administering a therapeutically effective amount of the compound to inhibit NO production in the patient.
42. The compound of any one of clauses 33 to 41, wherein the method comprises administering a therapeutically effective amount of the compound to inhibit iNOS gene expression in the patient.
43. The compound of any one of clauses 33 to 42, the method further comprising administering to the patient of one or more additional therapeutic agents.
44. The compound of clause 43, wherein the one or more additional therapeutic agents is an a corticosteroid, a salicylate, an acetic acid derivative, an enolic acid (oxicam) derivative, a propionic acid derivative, an anthranilic acid derivative, or a Cox-2 inhibitor.
45. The compound of clause 43, wherein the one or more additional therapeutic agents is selected from the group consisting of aspirin, diflunisal, salsalate, diclofenac, etodolac, indomethacin, ketorolac, nabumetone, sulindac, tolmetin, meloxicam, piroxicam, ibuprofen, naproxen, ketoprofen, fenoprofen, flurbiprofen, oxoprozin, mefenamic acid, celecoxib, cortisone, prednisone, and methylprednisone.

### BRIEF DESCRIPTION OF THE DRAWINGS

FIG. 1 is a chart showing the different effects of compound (I) and compound (II) on cell growth of primary human chondrocytes. Concentrations up to 5 mM were tested for each drug. IL-1β (IL-1B): 5 ng/mL.

FIG. 2 is a chart showing the comparison of NDX2 and NAG in IL-6 production in primary human chondrocytes. Concentrations of 1.25 mM and 2.5 mM were tested for each drug. IL-1β (IL-1B): 5 ng/mL.

FIGS. 3A-3B show the comparison of NDX2 and NAG on Cox-2 expression in primary human chondrocytes determined by western blot. FIG. 3A is a gel showing the effects of NDX2 and NAG on Cox-2 expression in primary human chondrocytes (Lane 1: NT; Lane 2: IL-1β; Lane 3: IL-1β+NDX2 at 2 mM; Lane 4: IL-1β+NAG at 2 mM). FIG. 3B is a chart that was obtained from a semi-quantitative analysis of the gel image. IL-1β: 5 ng/mL.

FIG. 4 is a chart showing the effect of NDX2 on mRNA levels of IL-6 in primary human chondrocytes. IL-1β (IL-1B): 5 ng/mL.

FIG. 5 is a chart showing the effect of NDX2 on mRNA levels of Cox-2 in primary human chondrocytes. IL-1β (IL-1B): 5 ng/mL.

FIG. 6 is a chart showing the effect of nitrite production in primary human chondrocytes. IL-1β (IL-1B): 5 ng/mL.

FIG. 7 is a chart showing the effect of NDX1 and NDX2 on cell growth of mouse macrophage cell line RAW264.7 cells. Concentrations up to 5 mM were tested for each drug.

FIG. 8 is a chart showing the comparison of NDX1, NDX2 and NAG in IL-6 production in RAW264.7 cells. Concentrations of up to 5 mM were tested for each drug. LPS: 100 ng/mL.

FIG. 9 is a gel image showing comparison of NDX1, NDX2 and NAG in iNOS and expression in mouse RAW264.7 macrophages by western blot. LPS: 100 ng/mL. Lane 1: NT; Lane 2: LPS; Lane 3: LPS+NDX1 at 0.1 mM; Lane 4: LPS+NDX1 at 1 mM; Lane 5: LPS+NDX2 at 0.5 mM; Lane 6: LPS+NDX2 at 5 mM; Lane 7: LPS+NAG at 0.5 mM; Lane 8: LPS+NAG at 5 mM.

FIG. 10 is a gel image showing comparison of NDX1, NDX2 and NAG in Cox-2 expression in mouse RAW264.7 macrophages by western blot. LPS: 100 ng/mL. Lane 1: NT; Lane 2: LPS; Lane 3: LPS+NDX1 at 0.1 mM; Lane 4: LPS+NDX1 at 1 mM; Lane 5: LPS+NDX2 at 0.5 mM; Lane 6: LPS+NDX2 at 5 mM; Lane 7: LPS+NAG at 0.5 mM; Lane 8: LPS+NAG at 5 mM.

FIG. 11 is a chart showing comparison of NDX1, NDX2 and NAG in iNOS expression in mouse RAW264.7 macrophages determined by RT-PCR analysis. LPS: 100 ng/mL.

FIG. 12 is a chart showing comparison of NDX1, NDX2 and NAG in IL1β (IL1B) expression in mouse RAW264.7 macrophages determined by RT-PCR analysis. LPS: 100 ng/mL.

FIG. 13 is a chart showing comparison of NDX1, NDX2 and NAG in nitrite production in LPS-activated mouse RAW264.7 macrophages. LPS: 100 ng/mL.

FIG. 14 is a chart showing comparison of MPO activities of the lung tissues from mice with various treatment. LPS: 10 mg/kg, I.P.; NDX1, NDX2 & NAG: 200 mg/kg, I.V. N=8.

FIG. 15 is a chart showing comparison of leukocytes immigration to the peritoneal cavity from mice with various treatments. LPS: 10 mg/kg, I.P.; NDX1, NDX2 & NAG: 300 mg/kg, I.V. N=8.

FIGS. 16A-16D show the comparison of leukocytes immigration to the peritoneal cavity from mice with various treatments. FIG. 16A shows an image of leukocytes immigration to the peritoneal cavity from mice with treatment of LPS: 10 mg/kg, I.P.; NAG: 200 mg/kg, I.V. N=8. White arrow: neutrophils; Red arrow: monocytes. FIG. 16B shows an image of leukocytes immigration to the peritoneal cavity from mice with treatment of LPS: 10 mg/kg, I.P.; NDX1: 200 mg/kg, I.V. N=8. White arrow: neutrophils; Red arrow: monocytes. FIG. 16C shows an image of leukocytes immigration to the peritoneal cavity from mice with treatment of LPS: 10 mg/kg, I.P.; NDX2: 200 mg/kg, I.V. N=8. White arrow: neutrophils; Red arrow: monocytes.

FIG. 16D shows a chart showing comparison of leukocytes immigration to the peritoneal cavity from mice with various treatments. LPS: 10 mg/kg, I.P.; NDX1, NDX2 & NAG: 200 mg/kg, I.V. N=8.

FIG. 17 is a chart showing comparison of the serum IL6 levels in mice with various treatments. LPS: 10 mg/kg, I.P.; NDX1, NDX2 & NAG: 300 mg/kg, I.V. N=8.

FIG. 18 is a chart showing comparison of the serum TNFa levels in mice with various treatments. LPS: 10 mg/kg, I.P.; NDX1, NDX2 & NAG: 300 mg/kg, I.V. N=8.

FIG. 19 is a chart showing comparison of the serum IL6 levels in mice with various treatments. LPS: 10 mg/kg, I.P.; NDX1, NDX2 & NAG: 200 mg/kg, I.V. N=8.

FIG. 20 is a chart showing comparison of the serum TNFa levels in mice with various treatments. LPS: 10 mg/kg, I.P.; NDX1, NDX2 & NAG: 200 mg/kg, I.V. N=8.

FIG. 21 is a chart showing comparison of the effects of NDX1, NDX2 and NAG on cell growth of primary mouse peritoneal macrophages. Concentrations up to 1 mM were tested for each drug. No drugs were added to the non-treatment group (NT). There were eight repeats in each group (n=8). *P<0.05 vs LPS group. The experiment was duplicated.

FIG. 22 is a chart showing comparison of NDX1, NDX2, and NAG in IL-6 production in primary mouse peritoneal macrophages. Various concentrations (0.01 mM, 0.1 mM and 1 mM were tested for NDX1 and NDX2, while NAG at 0.1 mM and 1 mM was used as control. LPS: 100 ng/mL. There were eight repeats in each group (n=8). *P<0.05 vs LPS group. The experiment was duplicated.

FIG. 23 is a chart showing comparison of NDX1, NDX2, and NAG in TNF α production in primary mouse peritoneal macrophages. Various concentrations (0.01 mM, 0.1 mM and 1 mM were tested for NDX1 and NDX2, while NAG at 0.1 mM and 1 mM was used as control. LPS: 100 ng/mL. There were eight repeats in each group (n=8). *P<0.05 vs LPS group. The experiment was duplicated.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination.

### Representative Embodiments

In some embodiments, the disclosure provides a method of treating a subject having an inflammatory disease, comprising administering a therapeutically effective amount of a compound of the formula

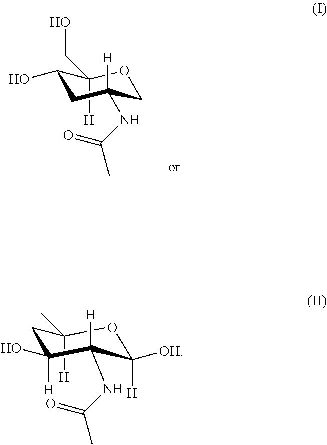

In some embodiments, the disclosure provides the use a compound of the formula

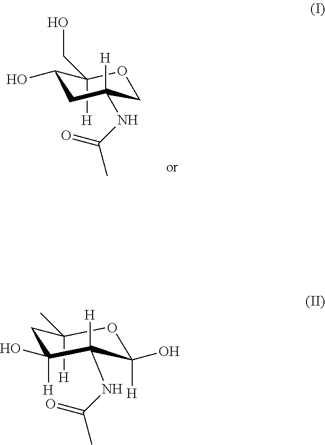

or a pharmaceutically acceptable salt thereof, in the preparation of a medicament for treating a subject having an inflammatory disease. In some embodiments, the disclosure provides a pharmaceutical composition comprising a compound of the formula

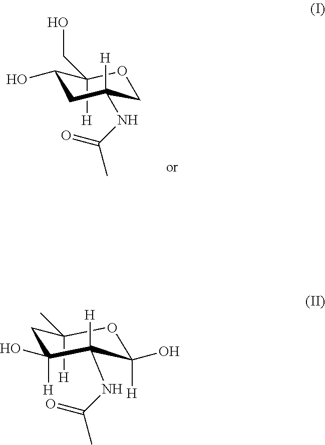

or a pharmaceutically acceptable salt thereof, and optionally a pharmaceutically acceptable carrier or excipient.

As used herein, the term “subject” or “patient” refers to a human or, in the case of veterinary applications, can be a laboratory, agricultural, domestic, or wild animal. The methods described herein can be applied to subjects including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats.

As used herein, the term “therapeutically effective amount” refers to an amount of a drug or agent that elicits the biological or medicinal response in a subject (i.e. a tissue system, animal or human) that is being sought by a researcher, veterinarian, medical doctor or other clinician, which includes, but is not limited to, alleviation of the symptoms of the inflammatory disease being treated. In some embodiments, the therapeutically effective amount is that amount of an active which may treat or alleviate the inflammatory disease or symptoms of the inflammatory disease at a reasonable benefit/risk ratio applicable to any medical treatment. In some embodiments, the therapeutically effective amount is that amount of an active which may act as a disease modifying drug for the inflammatory disease being treated. In some embodiments, the therapeutically effective amount is that amount of an inactive prodrug which when converted through normal metabolic processes produces an amount of active drug capable of eliciting the biological or medicinal response in a subject that is being sought. In some embodiments, the therapeutically effective amount is that amount of an active which may bring about a change in the activity of a biological target in the subject administered the active. In some embodiments, the therapeutically effective amount is that amount of an active which may inhibit NO production in the patient. In some embodiments, the therapeutically effective amount is that amount of an active which may inhibit iNOS gene expression in a subject.

It will be appreciated that inflammation and inflammatory processes can occur in connection with a wide-range diseases and disease states. It will be appreciated that there are two types of inflammation: acute and chronic. While acute inflammation can be beneficial in that it can indicate a subject is fighting infection and/or can help speed up the healing process from an injury or illness, chronic inflammation that can result from the immune system sending chemical messengers that drive inflammatory processes for a prolonged amount of time can cause debilitating pain and illness. It will be appreciated that chronic inflammation is associated with heart disease, diabetes, cancer, arthritis, and bowel diseases such as Crohn's disease and ulcerative colitis. In some embodiments, a disease to be treated in connections with the present disclosure can be any disease in which the pro-inflammatory mediator nitric oxide (NO) interacts with tissue in a subject to generate pathogenic effects and/or drive a disease. In some embodiments, the disease in which the pro-inflammatory mediator nitric oxide (NO) can act include neurodegeneration in numerous diseases of the nervous system, including Parkinson's disease (PD), Alzheimer's disease (AD), amyotrophic lateral sclerosis (ALS), Huntington's disease (HD), and ischemic brain injury (stroke). In some embodiments, a disease to be treated in connections with the present disclosure can be any disease in which the aberrant expression of the iNOS gene plays a role the development of the disease. Exemplary diseases include inflammatory diseases such as rheumatoid arthritis (RA), osteoarthritis (OA), colitis, asthma, multiple sclerosis, systemic sclerosis, Sjögren's syndrome, bronchiectasis, idiopathic pulmonary fibrosis, atherosclerotic plaques, ulcerative colitis, Crohn's disease, necrotizing enterocolitis, Coeliac disease, glomerulonephritis, dialated cardiomyopathy, cutaneous lupus erythematosus, systemic lupus erythematosus, dermatitis, periapical periodontitis, psoriasis, and the like. In some embodiments, the inflammatory disease can be rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like.

It will be appreciated that the methods, uses, compositions, or compounds described herein can be administered in any of the modes of administration known in the art. As used herein, “administering” or “administered” includes all means of introducing the compounds and compositions described herein to a subject, including, but are not limited to, oral (po), intravenous (iv), intramuscular (im), subcutaneous (sc), transdermal, inhalation, buccal, ocular, sublingual, nasal, vaginal, rectal, and the like. The methods, uses, compositions, or compounds described herein may be administered in unit dosage forms and/or formulations containing conventional nontoxic pharmaceutically-acceptable carriers, adjuvants, and/or vehicles.

In some embodiments, the methods, uses, compositions, or compounds described herein can be administered orally. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Solid formulations for oral administration may be formulated to be immediate and/or modified release formulations. Modified release formulations include delayed, sustained, pulsed, controlled, targeted and programmed release formulations.

In some embodiments, the methods, uses, compositions, or compounds described herein can be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous means of administration.

In some embodiments, the methods, uses, compositions, or compounds described herein can be co-administered or co-formulated with one or more additional therapeutic agents. In some embodiments, the one or more additional therapeutic agents is an a corticosteroid, a salicylate, an acetic acid derivative, an enolic acid (oxicam) derivative, a propionic acid derivative, an anthranilic acid derivative, or a Cox-2 inhibitor. In some embodiments, the one or more additional therapeutic agents is selected from the group consisting of aspirin, diflunisal, salsalate, diclofenac, etodolac, indomethacin, ketorolac, nabumetone, sulindac, tolmetin, meloxicam, piroxicam, ibuprofen, naproxen, ketoprofen, fenoprofen, flurbiprofen, oxoprozin, mefenamic acid, celecoxib, cortisone, prednisone, and methylprednisone.

Any effective regimen for administering the compounds and compositions described herein can be used. For example, compounds and compositions described herein can be administered as single doses, or the doses can be divided and administered as a multiple-dose daily regimen. Further, a staggered regimen, for example, one to five days per week can be used as an alternative to daily treatment. In some embodiments, a subject is administered multiple doses in the methods, uses, compounds, or compositions described herein. In some embodiments, a subjected is administered multiple doses (preferably about 2 up to about 80 doses) with a compound or composition as described herein, for example, at 8-72 hour intervals or at 8-12 hour intervals.

Any suitable course of therapy with the compound of the formula (I) or (II) described herein can be used. In one embodiment, individual doses and dosage regimens are selected to provide a total dose administered during a given day of about 1 mg to about 1000 mg mg; or about 200 mg to about 1000 mg. In some embodiments, the compound of the formula (I) or (II) is administered in the methods or uses described herein in a single daily dose (QD), or in a twice daily dose (BID), or a three times daily dose (TID). In some embodiments, the compound of the formula (I) or (II) is administered in the methods or uses described herein in a twice daily dose (BID) at a dose of about 300 mg to about 900 mg per dose. In some embodiments, the compound of the formula (I) or (II) is administered in the methods or uses described herein in a twice daily dose (BID) at a dose of about 600 mg to about 800 mg. In some embodiments, the compound of the formula (I) or (II) is administered in the methods or uses described herein in a twice daily dose (BID) at a dose of about 700 mg. In some embodiments, the compound of the formula (I) or (II) is administered in the methods or uses described herein in cycles lasting days a week, 2 weeks, 3 weeks, 4 weeks, 5 weeks, and the like. In some embodiments, the compound of the formula (I) or (II) is administered daily in the methods or uses described herein for between 10 and 45 days, or until cessation of treatment is indicated by patient status as observed by a treating physician. In some embodiments, the compound of the formula (I) or (II) is administered daily in the methods or uses described herein for between 10 and 20 days, or until cessation of treatment is indicated by patient status as observed by a treating physician. In some embodiments, the compound of the formula (I) or (II) is administered daily in the methods or uses described herein for between 25 and 35 days, or until cessation of treatment is indicated by patient status as observed by a treating physician. In some embodiments, the compound of the formula (I) or (II) is administered daily in the methods or uses described herein for about 30 days, or until cessation of treatment is indicated by patient status as observed by a treating physician.

It will be appreciated that the unitary daily dosage of the compound of the formula (I) or (II) can vary significantly depending on the patient condition, the inflammatory disease being treated, the route of administration of the compound of the formula (I) or (II), and the possibility of co-administration of additional therapeutic agents, as described herein. The effective amount to be administered to a patient is based on body surface area, mass, and physician assessment of patient condition.

The examples and preparations provided below further illustrate and exemplify particular aspects of embodiments of the disclosure. It is to be understood that the scope of the present disclosure is not limited in any way by the scope of the following examples. The compounds of the formula (I) and (II) (also referred to as NDX1 and NDX2 respectively can be prepared according to the methods described in WO2022/051575, the entire contents of which is incorporated herein by reference. N-acetyl glucosamine (a.k.a. NAG) can be purchased from commercial sources, such as Sigma-Aldrich (St. Louis, MO).

### Example 1: To Determine the Non-Toxic Dose Ranges of NDXs for the Human Chondrocyte Cultures

### Primary Cultures of Human Chondrocytes:

Normal cartilage tissues. Articular cartilages were harvested from the femoral condyles and the tibial plateaus, which were obtained from autopsy services at the hospital of UVA and approved by the Institutional Review Board. All tissue samples were graded according to a modified Mankin scale, and only cartilages without evidence of osteoarthritis were used as a source of chondrocytes. The interval between death and the time the cartilages were harvested from these knee joints in the laboratory were at least 24 h and ranged up to 96 h. Cartilage shavings were harvested at the operation room and placed in tissue culture medium (DMEM, 10% FBS, penicillin, streptomycin), and shipped to the laboratory at 4° C. The tissues were processed in the laboratory within 24 h after harvest.

Chondrocytes were isolated from the cartilage by collagenase digestion and maintained in continuous monolayer cultures in DMEM containing 10% FBS. Cell viability after chondrocyte isolation by collagenase digestion of normal cartilage was >95%, determined by the typan blue stain (Sigma-Aldrich). Experiments were performed with primary or first passage cells. When necessary, trypsin will be used for cell passage.

### Cell Proliferation Assays

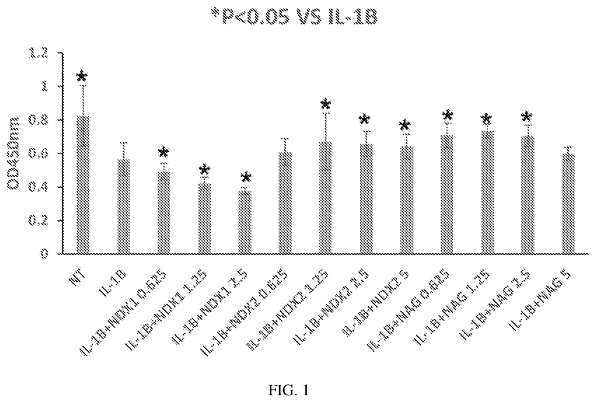

Human chondrocytes were seeded in a 96-well plate (5×104/well) and incubated with culture medium overnight. Cells were then treated as needed and the Roche-Cell Proliferation Reagent WST-1 (Fisher Scientific Company, Nazareth, PA) were used for cell proliferation assays by following the enclosed instructions provided by the manufacturer. No IL-1β and the other drugs were added in the non-treatment group (NT). There were six repeats in each group (n=6). The experiment was duplicated. The results are shown in FIG. 1.

The WST-1 assay showed that NDX1 was dose-dependently toxic against human chondrocytes in the presence of IL-1β (IL-1B). NDX1 showed remarkable cytotoxicity. NDX2 and NAG could prevent inhibition of cell proliferation induced by IL-1β (n=6).

### Data Analysis:

The obtained values were presented as means±SD and analyzed using one-way ANOVA followed by the Bonferroni/Dunnet's test. The level of statistical significance between two groups was set at P<0.05.

### Example 2: To Assess the Preventive Effects of NDXs on IL-1β Activation of Human Chondrocyte Cultures

### Cell Treatment

Primary cultures of human chondrocytes were seeded in a 96-well plate (5×104/well) and treated by different drugs for 24 h. Both culture media and cells were collected for the following assays.

### Detection of IL-6 Level

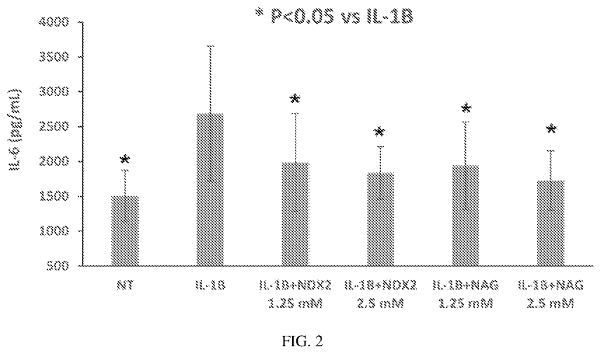

The amount of IL-6 in the culture medium were measured by a commercial ELISA kit (Sigma-Aldrich), following the instructions provided by the manufacturer. Briefly, the culture media were mixed with different assay reagents step by step. The resultant solutions were read at 450 nm on a microplate reader. The ELISA test showed that NDX2 significantly inhibited production of IL-6 in IL-1B stimulated human chondrocytes, with its activity comparable to NAG (n=6). No IL-1β and the other drugs were added in the non-treatment group (NT). The experiment was duplicated. Results are shown in FIG. 2.

### Western Blot Analysis

The proteins were prepared from cells growing on a 6-well plate (approximately 500,000/well) and the protein concentration were determined using the Bradford protein assay kit (Bio-Rad, Hercules, CA, USA). The samples containing 100 μg proteins were run on 10% SDS-polyacrylamide gels at a constant current of 80 V and electro-transferred to nitrocellulose membranes (Thermo Scientific) at a constant voltage of 10 V overnight. Membranes were blocked with 5% fatty acid free bovine serum albumin (BSA) fraction V (Roche Diagnostics, Indianapolis, IN, USA) in TBST solution (50 mM Tris, pH 7.6, 150 mM NaCl, 0.05% Tween 20) for 1 hr at RT, washed, and incubated overnight at 4° C. in 5% BSA in TBST solution containing each of the specific primary antibodies against COX-2 (Santa Cruz Biotechnology). β-actin was used as the loading control. The membranes were then incubated with the corresponding horseradish peroxidase (HRP)-conjugated secondary antibody (Cell Signaling) for 1 hr at RT followed by a chemiluminescent substrate for HRP antibody and enhancer solution (Thermo Scientific) mixed in a 1:1 ratio. The membranes were incubated in the dark with autoradiography films (Genesee Scientific, San Diego, CA, USA), and the films were developed to visualize the bands. Then the grayscale images (300-400 dpi) on films were scanned using a flat-bed scanner. Densitometry were carried out in Photoshop. Using the magic wand tool from the tool palette, the area of each band was selected, and the mean histogram was recorded and then charted. No IL-1β and the other drugs were added in the non-treatment group (NT). The experiment was duplicated.

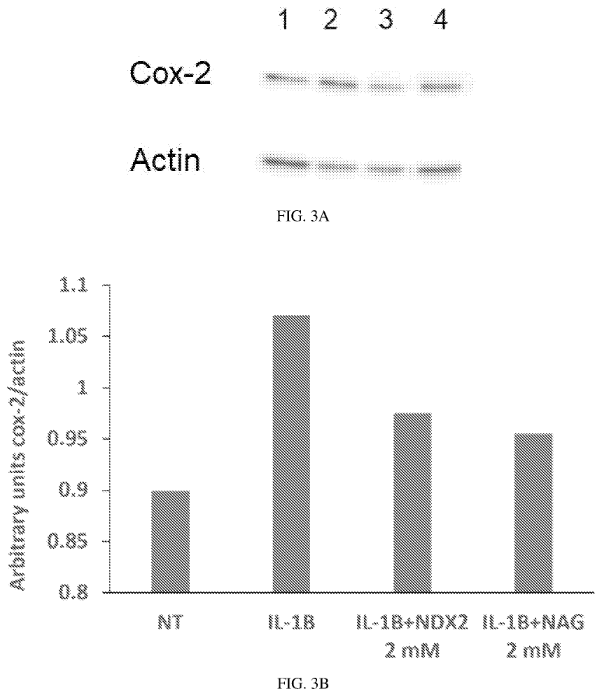

The Western blot showed that NDX2 significantly inhibited Cox-2 expression in IL-1B stimulated human chondrocytes, with its activity comparable to NAG. Results are shown in FIGS. 3A and 3B.

### Gene Expression Analysis

Total RNA was extracted and purified from cells using an RNeasy kit (QIAGEN Sciences, Valencia, CA) according to the protocol provided by the manufacturer. The RNase-Free DNase was used to digest DNA during RNA purification. Synthesis of cDNA from total RNA and the quantitative PCR was carried out by using the iScript™ cDNA synthesis kit and the iQ™ SYBR Green Supermix kit (Bio-Rad Laboratories, Hercules, CA), respectively. The target genes included COX-2 and IL-6. Gene of 18s ribosomal RNA was used as an internal control. The primer sequences were listed as follows: 5′-GTGACCAGTTCACTCTTGGT-3′ (forward) (SEQ ID NO:1), 5′-CATTGGAAGTGAAGCGTTTCG-3′ (reverse) (SEQ ID NO:2) for 18s rRNA, 5′-TGCATTCTTTGCCCAGCACT-3′ (forward) (SEQ ID NO:3), 5′-AAAGGCGCAGTTTACGCTGT-3′ (reverse) (SEQ ID NO:4) for Cox-2, and 5′-GGTACATCCTCGACGGCATCT-3′ (forward) (SEQ ID NO:5), 5′-GTGCCTCTTTGCTGCTTTCAC-3′ (reverse) (SEQ ID NO:6) for IL-6. The threshold cycle (CT) value was calculated from amplification plots. Data was analyzed using the 2-ΔΔCT method with 18s rRNA serving as the reference. Gene expression was normalized to the control group in each experiment and represented as fold of change. No IL-1β and the other drugs were added in the non-treatment group (NT). There were four repeats in each group (n=4). The experiment was duplicated.

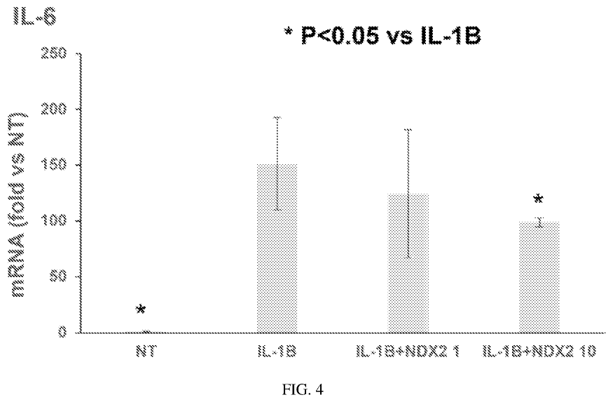

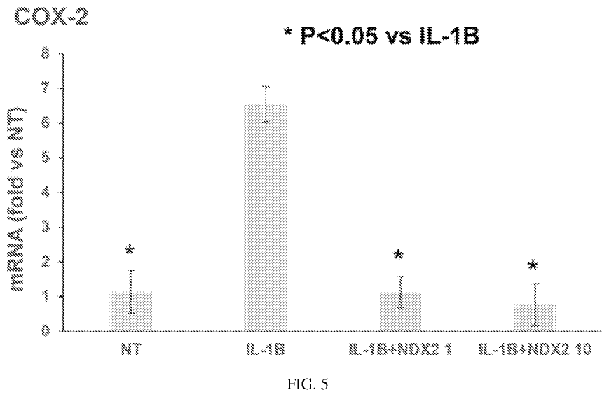

The RT-PCR analysis showed that NDX2 could inhibit increase in mRNA level of pro-inflammatory genes IL-6 and COX-2 induced by IL-1β (n=4). Results are shown in FIGS. 4 and 5.

### Detection of Nitrite Production

The Griess reagent system is based on the chemical reaction which uses sulfanilamide and N-1-napthylethylenediamine dihydrochloride (NED) under acidic (phosphoric acid) conditions. This system detects nitrite production. In the present experiments, nitric oxide detection will be performed by a commercial kit (Promega Corporation, Madison, WI, USA), according to the manufacturer's manual. Briefly, all experimental samples (culture medium in each group) and standards containing the dilution series for the nitrite standard reference curve were mixed with 50 μL of the sulfanilamide solution and incubated for 10 minutes at room temperature without light in a 96-well plate. Then 50 μl of the NED Solution was added to each well and incubated 10 minutes at room temperature without light. The optical density (OD) was determined at 530 nm on a microplate reader within 30 minutes. No IL-1β and the other drugs were added in the non-treatment group (NT). There were six repeats in each group (n=6). The experiment was duplicated.

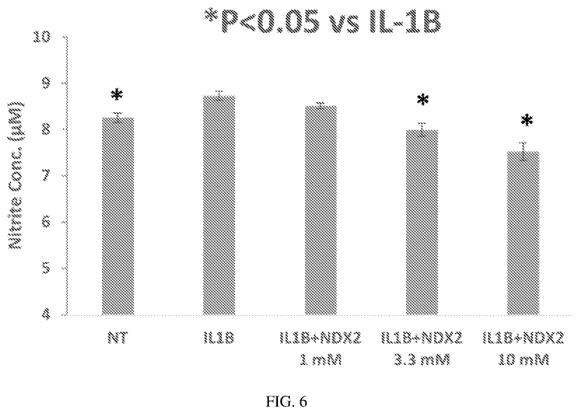

Using the Griess reagents, it was found that NDX2 dose-dependently inhibited production of nitric oxide in IL-1β stimulated human chondrocytes (n=6). Results are shown in FIG. 6.

### Data Analysis

The obtained values were presented as means±SD and analyzed using one-way ANOVA followed by the Bonferroni/Dunnet's test. The level of statistical significance between two groups was set at P<0.05.

### Example 3: Mouse Macrophage Assays to Determine the Non-Toxic Dose Ranges of NDXs for the Mouse Macrophages RAW264.7 Cells

### Cell Culture and Treatment

Mouse RAW264.7 macrophage cell line (ATCC; Manassas, VA, USA) was maintained in DMEM culture medium containing 10% FBS and 1% antibiotic mix. For cell treatment, the test drugs at different doses were added to the culture 0.5 h prior to addition of 100 ng/mL LPS (Sigma-Aldrich Co., St Louis, MO, USA) and then cells were co-treated with LPS for 24 hours.

### Cytotoxicity Assay

Macrophages in the control group and the treatment groups was seeded on a 96-well plate for 24 hrs and the cell number was counted using the WST-1 kit (Fisher Scientific). After removal of the supernatant, 150 μL of the fresh medium was added into each well. Then 15 μL of the Cell Proliferation Reagent WST-1 was added and incubated with cells for 3 hrs in dark with a humidified atmosphere of 5% carbon dioxide at 37° C. Each sample was determined at 450 nm on a microplate reader. No drugs were added in the non-treatment group (NT). There were eight repeats in each group (n=8). The experiment was duplicated.

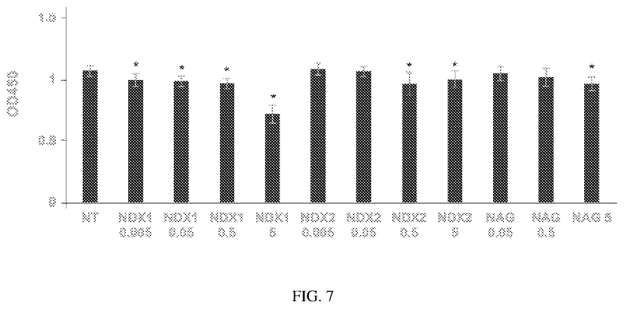

The WST-1 assay showed that NDX1 was dose-dependently toxic against mouse macrophage cell line RAW264.7 cells; NDX2 and NAG showed less toxicity than NDX1 (n=8). Results are shown in FIG. 7.

### Statistical Analysis

Data was expressed as mean±SD. Statistical evaluation was performed by the Analysis Of Variance (ANOVA) using the SPSS 15.0 software. A student t-test (two-tailed) was performed to compare the difference between the two groups, and p<0.05 was considered significant.

### Example 4: To Assess Effects of NDXs on Mouse Macrophage RAW264.7 Cells Activated by LPS

### Detection of IL-6 Level

The amounts of IL-6 in culture medium from different treatments were measured by a commercial ELISA kit (Sigma-Aldrich), following the instructions provided by the manufacturer. Briefly, cells were seeded on a 96-well plate and various treatments were performed. The culture medium were collected and mixed with different assay reagents step by step. The resultant solutions were read at 450 nm on a microplate reader. There were eight repeats in each group (n=8). *P<0.05 vs LPS group. The experiment was duplicated.

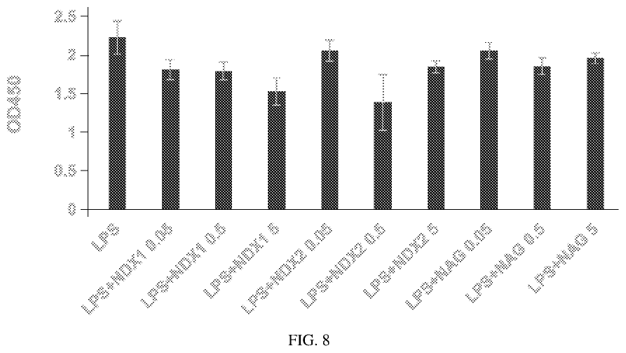

The ELISA test showed that all three drugs tested (NDX1, NDX2 and NAG) could significantly reduce IL-6 production in LPS-activated mouse RAW264.7 macrophages, while NDX1 treatment was most effective among them (n=8). Results are shown in FIG. 8.

### Western Blot Analysis

The cellular proteins were prepared from cells growing on a 6-well plate (approximately 500,000/well) using a commercial cell lysis buffer (Thermo Scientific). Then the protein concentration was determined using the Bradford protein assay kit (Bio-Rad, Hercules, CA, USA). The samples containing 100 μg proteins were run on 10% SDS-polyacrylamide gels at a constant current of 80 V and electro-transferred to nitrocellulose membranes (Thermo Scientific) at a constant voltage of 10 V overnight. Membranes were blocked with 5% fatty acid free bovine serum albumin (BSA) fraction V (Roche Diagnostics, Indianapolis, IN, USA) in TBST solution (50 mM Tris, pH 7.6, 150 mM NaCl, 0.05% Tween 20) for 1 hr at RT, washed, and incubated overnight at 4° C. in 5% BSA in TBST solution containing each of the specific primary antibodies against mouse iNOS and Cox-2 (both from Novus Biologicals, LLC). β-actin was used as the loading control. The membranes were then incubated with the corresponding horseradish peroxidase (HRP)-conjugated secondary antibody (Cell Signaling) for 1 hr at RT followed by a chemiluminescent substrate for HRP antibody and enhancer solution (Thermo Scientific) mixed in a 1:1 ratio. The membranes were incubated in the dark with autoradiography films (Genesee Scientific, San Diego, CA, USA), and the films were developed to visualize the bands. Then the grayscale images (300-400 dpi) on films were scanned using a flat-bed scanner. Densitometry was carried out in Photoshop. Using the magic wand tool from the tool palette, the area of each band was selected, and the mean histogram were recorded and then charted. All Western blots were conducted in duplicates. No LPS and the other drugs were added in the non-treatment group (NT).

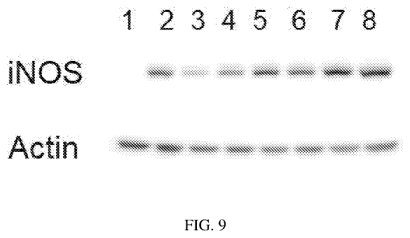

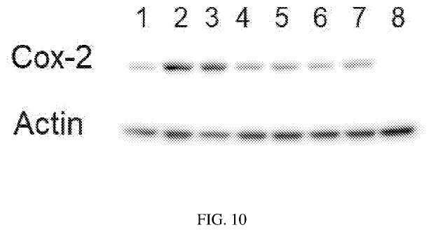

The Western blot assay revealed that NAG>NDX2>NDX1 for inhibition of Cox-2 expression and NDX1>NDX2>NAG for inhibition of iNOS expression, respectively. Results are shown in FIGS. 9-10.

### Gene Expression Analysis

Total RNA was purified using an RNeasy kit (QIAGEN Sciences, Valencia, CA) according to the protocol provided by the manufacturer, and was stored at −80° C. Synthesis of cDNA from total RNA and the quantitative PCR were carried out by using the iScript™ cDNA synthesis kit and the iQ™ SYBR Green Supermix kit (Bio-Rad Laboratories, Hercules, CA), respectively. The target genes included iNOS, IL-6 and IL-1β. Gene of 18s ribosomal RNA was used as an internal control. The threshold cycle (CT) value was calculated from amplification plots. Data was analyzed using the 2-ΔΔCT method with 18s rRNA serving as the reference. Gene expression was normalized to the control group in each experiment and represented as fold of change. The primer sequences were listed as follows: 5′-GTGACCAGTTCACTCTTGGT-3′ (forward) (SEQ ID NO:1), 5′-CATTGGAAGTGAAGCGTTTCG-3′ (reverse) (SEQ ID NO:2) for 18s rRNA, 5′-GAGGGATGCCTTCCGCAGCTG-3′ (forward) (SEQ ID NO:7), 5′-GAATCGAACCCTGATTCCCCGTC-3′ (reverse) (SEQ ID NO:8) for iNOS, 5′-CAACCAACAAGTGATATTCTCCATG-3′ (forward) (SEQ ID NO:9), 5′-GATCCACACTCTCCAGCTGCA-3′ (reverse) (SEQ ID NO:10) for IL-1β, and 5′-GAGTCCTTCAGAGAGATACAG-3′ (forward) (SEQ ID NO: 11), 5′-TGGTCTTGGTCCTTAGCC-3′ (reverse) (SEQ ID NO:12) for IL-6.

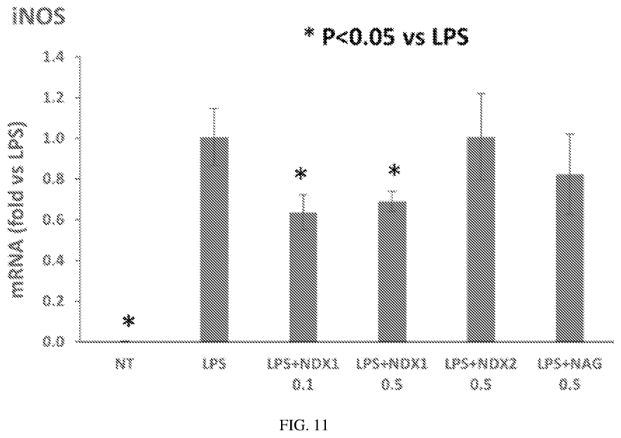

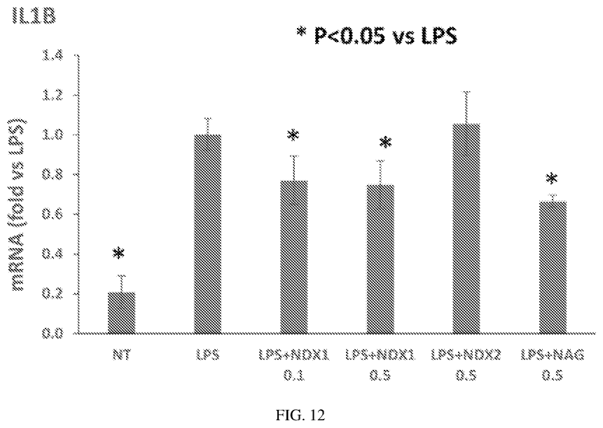

The RT-PCR showed that NDX1 (0.1 mM and 0.5 mM) could inhibit mRNA expression of both iNOS and IL1β, while NAG (0.5 mM) could inhibit IL1β but not iNOS mRNA expression. NDX2 (0.5 mM) didn't inhibit either iNOS or IL1β mRNA expression (n=4). Results are shown in FIGS. 11-12.

### Detection of Nitrite Production

The Griess reagent system is based on the chemical reaction which uses sulfanilamide and N-1-napthylethylenediamine dihydrochloride (NED) under acidic (phosphoric acid) conditions. This system detects nitrite production. In the present experiments, nitric oxide detection was performed by a commercial kit (Promega Corporation, Madison, WI, USA), according to the manufacturer's manual. Briefly, all experimental samples and standards containing the dilution series for the nitrite standard reference curve were mixed with 50 μL of the sulfanilamide solution and incubated 10 mins at room temperature without light in a 96-well plate. Then 50 μL of the NED Solution was added to each well and incubated 10 mins at room temperature without light. The optical density (OD) was determined at 530 nm on a microplate reader within 30 mins. No LPS and the other drugs were added in the non-treatment group (NT). The experiment was duplicated. There were six repeats in each group (n=6). The experiment was duplicated.

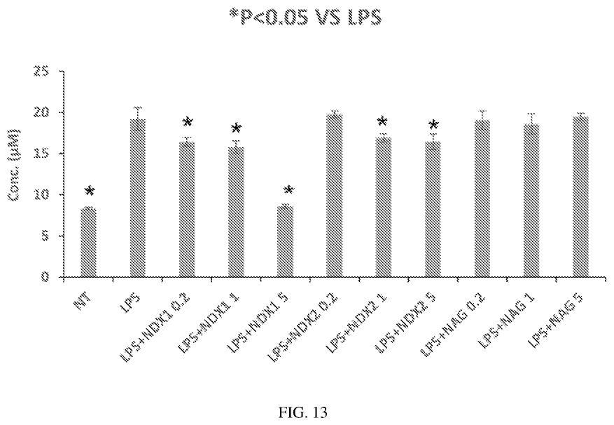

The nitrite test showed that NDX1 (0.2 mM, 1 mM and 5 mM) and NDX2 (0.5 mM and 5 mM) could significantly inhibit nitric oxide production (n=6), while NAG (0.5-5 mM) couldn't. Results are shown in FIG. 13.

### Example 5: To Determine the Anti-Inflammatory Activities of NDX1 and NDX2 Against LPS-Induced Systemic Inflammation in Mice

### Animals

Male C57BL6/J mice (8 to 10-week-old, 20-25 g) purchased from Charles River were used in this study. Mice were kept in the animal facility of the School of Medicine, University of Virginia (UVa). All experimental procedures were approved by the Ethics Committee on Animal Research of Medical School, UVa and are in accordance with the Guidelines of the National Council for Animal Experimentation Control (CONCEA).

### Animal Model

Moderate endotoxemia by LPS was induced with a single dose (10 mg/Kg, i.p.) of LPS (Escherichia coli 0111:B4, Sigma Chemical Co., St. Louis, MO, United States), 30 min after intravenous injection of NAG or NDX1 or NDX2 or the vehicle (saline). Two doses (200 mg/kg body weight and 300 mg/kg body weight) were tested for NAG, NDX1 and NDX2. Animals without any treatment were used to obtain the baseline data. Six hours after LPS treatment, inflammation assays were performed as described below. A total of 40 animals were randomly divided into 5 groups (8 mice/group): non-treatment (NT); 10 mg/Kg LPS plus saline (LPS); 10 mg/Kg LPS plus NAG (LPS+NAG); 10 mg/Kg LPS plus NDX1 (LPS+NDX1); and 10 mg/Kg LPS plus NDX2 (LPS+NDX2). The experiment was duplicated.

### Leukocyte Migration to the Peritoneal Cavity

Peritoneal cells were collected by lavage with ice-cold Dulbecco's modified Eagle's medium (DMEM, Gibco BRL, Gaithersburg, MD). Two methods were used: 1) the peritoneal cells were cultured for 24 h in DMEM supplemented with 10% FBS and antibiotics in a C02 incubator. Then WST-1 assay was performed to count cell number; 2) the peritoneal cells were fixed in 70% ethanol overnight at 4° C. for use. Cell suspension (100 uL) were then centrifuged onto glass covers of a 96 well plate at 1,500 rpm at 4° C. for 10 mins, stained with a fluorescent DNA dye DAPI which shows the cell nucleus, assessed by a Cytation 5 image reader and analyzed by the Gen5 software.

### Myeloperoxidase Activity Assay

Leukocyte migration to the lungs was evaluated using a myeloperoxidase kinetic-colorimetric assay. Tissue samples were collected in 50 mM K₂HPO₄ buffer (pH 6.0) containing 13.72 mM hexadecyltrimethylammonium bromide (HTAB) and stored at −80° C. until assayed. The samples were homogenized using a Tissue-Tearor, and homogenates were centrifuged (13.000 rpm, 2 min, 4° C.). Supernatants were assayed spectrophotometrically for myeloperoxidase activity at 450 nm.

### Leukocytes Migration to the Lung and the Peritoneal Cavity

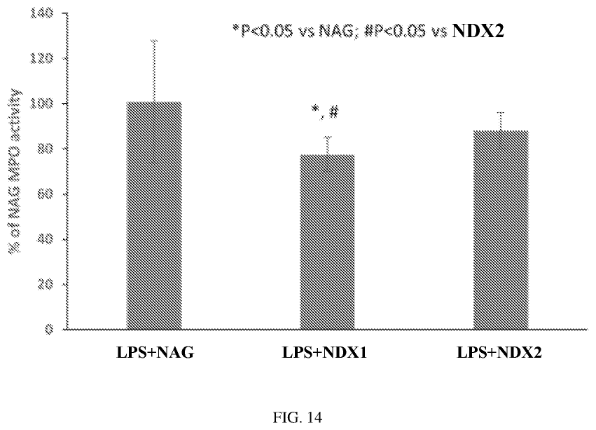

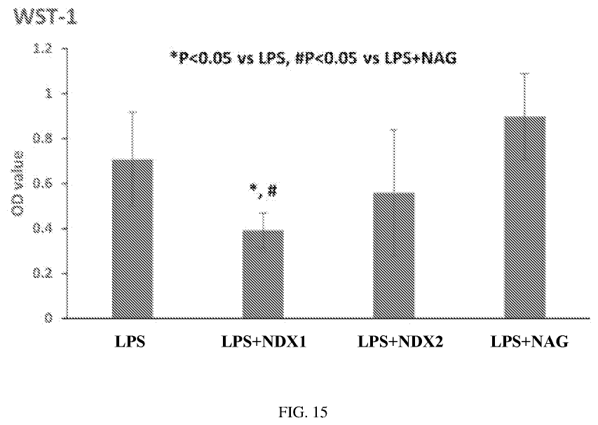

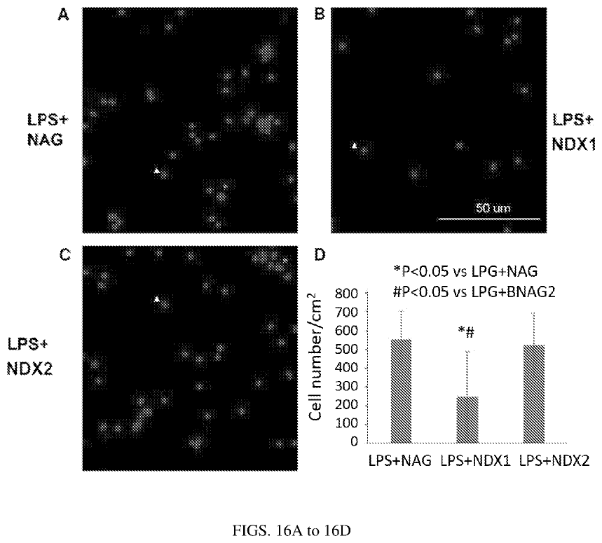

Sequestration of leukocytes from the circulation is an event that may compromise an appropriate response to infection. To compare the effects of NDX1 and NDX2 with NAG on leukocytes infiltration in the lungs, myeloperoxidase (MPO) activity was determined. Results shown in FIG. 14 demonstrate that NDX1 is the most effective in reducing leukocytes sequestration in the lung of LPS mice among the three compounds tested. In addition to reducing pulmonary leukocytes sequestration, the WST-1 test or DAPI staining may indicate that NDX1 together with LPS also significantly decreased leukocytes migration to the peritoneal cavity compared to the LPS alone, while neither NAG nor NDX2 did. The results indicate the sequestered leukocytes are mainly neutrophils (FIGS. 16A, 16B, 16C, white arrow) and a small number of monocytes which have a large size than neutrophils (FIGS. 16A, 16B, 16C, red arrow). Results are shown in FIGS. 15, 16A, 16B, 16C, and 16D.

### Determination of Cytokines Levels

TNFa and IL-6 concentrations in the serum were determined using enzyme-linked immunosorbent assay (ELISA) according to the manufacturer's instructions. Briefly, the serum from each animal was collected and mixed with different assay reagents step by step. The resultant solutions were read at 450 nm on a microplate reader.

In vivo experiments showed that NDX1 was the most effective in reducing inflammatory response to LPS in mice.

### Serum Levels of Inflammatory Cytokines

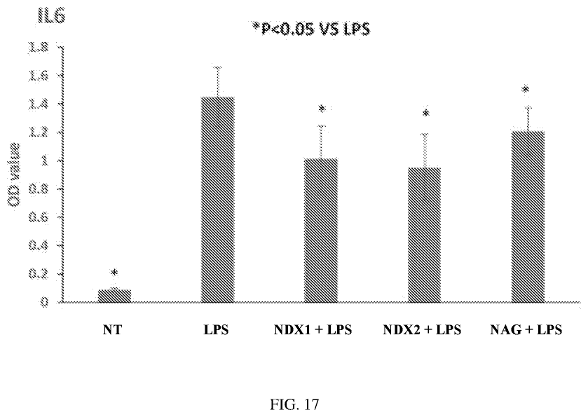

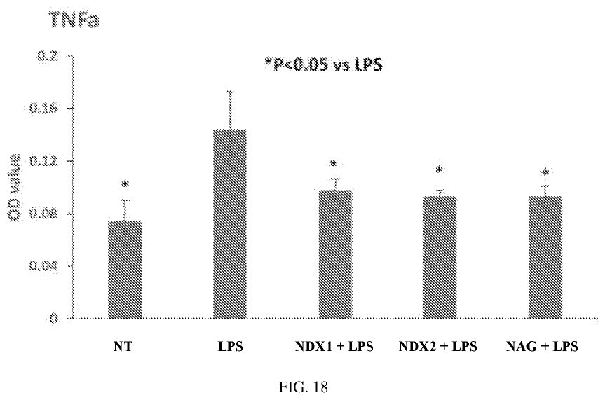

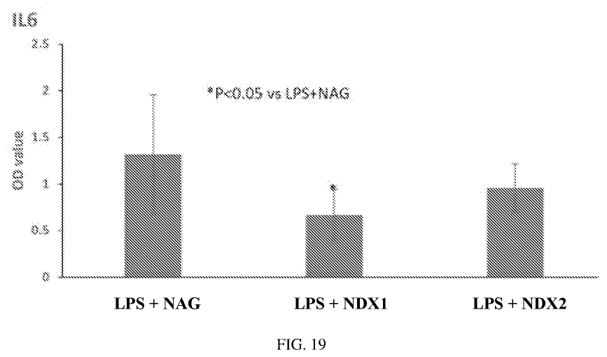

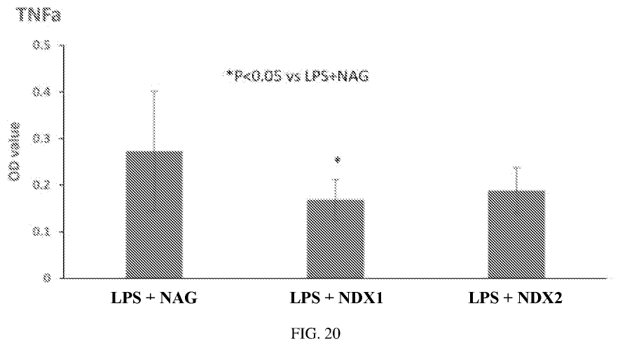

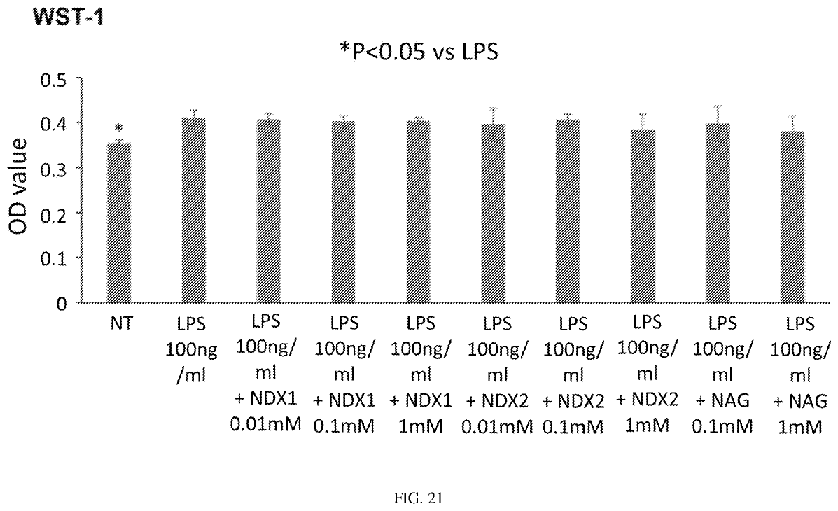

The production of inflammatory cytokines coordinates the response to infectious agents through the activation and recruitment of immune cells. To determine the production of inflammatory mediators, serum concentrations of IL-6 and TNFa were quantified. LPS increased serum levels of IL-6 and TNFa, and all three tested compounds NDX1, NDX2 and NAG at the dose of 300 mg/kg were able to significantly decrease serum levels of IL-6 and TNFa in LPS-mice. Results are shown in FIGS. 17, 18, 19, and 20. There was no significant difference in their effects among the three compounds at this dose. However, NDX1 showed significantly higher inhibitory activities against IL-6 and TNFa production than either NAG or NDX2 at a reduced dose of 200 mg/kg. Results are shown in FIGS. 21, 22, 23, and 24.

### Example 6: To Assess the Anti-Inflammatory Activities of NDX1 and NDX2 in LPS-Activated Primary Mouse Peritoneal Macrophages

### Experimental Groups

1. Non treatment (NT)
2. LPS 100 ng/mL
3. LPS 100 ng/mL+NDX1 dose1*
4. LPS 100 ng/mL+NDX1 dose2*
5. LPS 100 ng/mL+NDX1 dose3*
6. LPS 100 ng/mL+NDX2 dose1
7. LPS 100 ng/mL+NDX2 dose2
8. LPS 100 ng/mL+NDX2 dose3
9. LPS 100 ng/mL+NAG dose2
10. LPS 100 ng/mL+NAG dose3 (*dose 1, 2, 3 were selected as low, middle and high dose with no cytotoxicity from the cytotoxic assay)

### Cell Culture and WST-1 Assay

The peritoneal macrophages were isolated from peritoneal cavity, and cultured in Dulbecco's modified Eagle's medium (DMEM, Gibco BRL, Gaithersburg, MD) supplemented with 10% fetal bovine serum (FBS) (Hyclone Laboratories, Logan, VT) and 100 IU/mL penicillin G and 100 g/mL streptomycin, in a humidified atmosphere of 5% carbon dioxide at 37° C. Cells will grow on a 96-well plate and co-treated with 100 ng/ml LPS (Sigma-Aldrich) 0.5 hours after addition of BNAGs and NAG. Twenty-four hours later, the cell number was counted by using the WST-1 kit (Fisher Scientific). After removal of the supernatant, 150 l new medium was added into each cell. Then 15 μl Cell Proliferation Reagent WST-1 was added and incubated with cells for 3 hours in dark with a humidified atmosphere of 5% carbon dioxide at 37° C. Each sample was determined at 450 nm on a microplate reader.

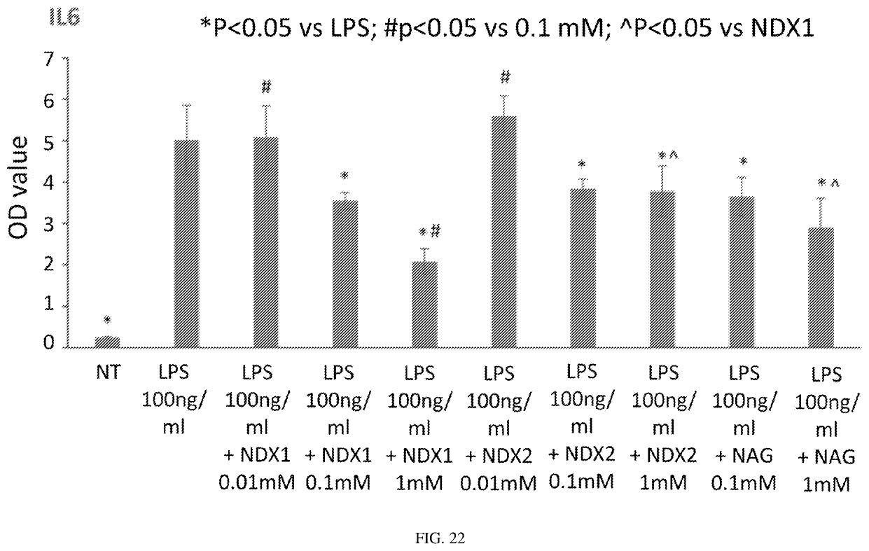

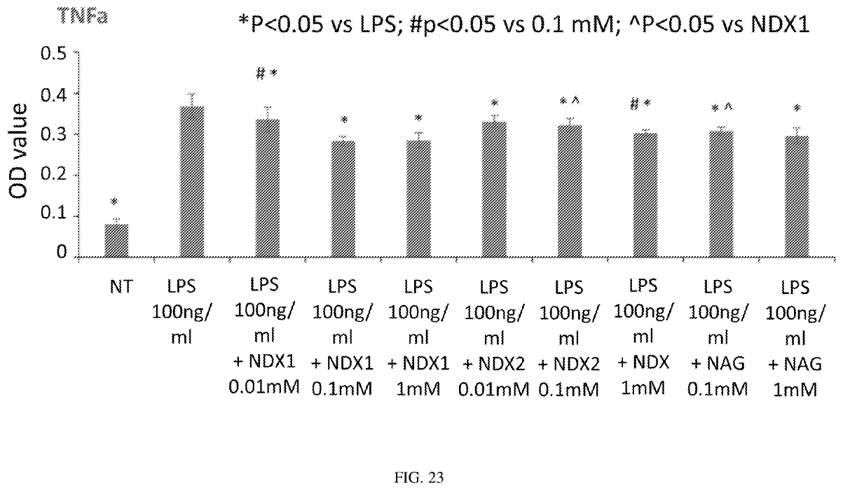

The WST-1 assay showed that LPS at 100 ng/mL significantly increased cell growth of mouse primary peritoneal macrophages. Each of the three tested compounds, NDX1, NDX2 or NAG at different doses of up to 1 mM combined with 100 ng/mL LPS, had no significant effect on cell number compared with LPS alone. Results are shown in FIG. 21. The ELISA tests showed that LPS at 100 ng/mL significantly increased IL6 and TNFa production in mouse primary peritoneal macrophages. All three drugs could significantly reduce IL-6 and TNFa production in LPS-activated macrophages, and NDX1 was the most effective among them. Results are shown in FIGS. 22 and 23.

### Detection of IL-6 and TNF α Level

The amounts of IL-6 and TNFα were measured by a commercial ELISA kit (Sigma-Aldrich), following the instructions provided by the manufacturer. Briefly, cells were seeded on a 96-well plate and various treatments were performed. The culture medium was collected and mixed with different assay reagents step by step. The resultant solutions were read at 450 nm on a microplate reader.

### Statistical Analysis

All data was reported as mean±SD. A one-way ANOVA test with multiple comparisons was performed. In each figure, the p-Values indicated if the difference between the selected control group compared with the rest of the samples was significant [P-value<0.05 (*)]. The GraphPad Prims program was used to obtain the results.

In vitro experiments showed that NDX1 was effective in reducing inflammatory response to LPS in mouse primary peritoneal macrophages.

### Example 7

In Examples 5 and 6, two compounds, NDX1 and NDX2, which are molecules obtained from modifications of NAG, were examined. Their anti-inflammatory properties to the parent molecule using an in vivo model of mouse systemic inflammation and an in vitro inflammation model of mouse primary peritoneal, both induced by LPS. While all three compounds at the tested dose of 300 mg/kg demonstrated significant inhibitory effect on the elevated serum levels of cytokines IL-6 and TNF α production caused by LPS, NDX1 showed the highest potency among the three compounds at the dose of 200 mg/kg. NDX1 also exhibited the strongest inhibition against the leukocyte immigration to the lung and peritoneal cavity induced by LPS. In vitro experiments revealed that NDX1, NDX2 and NAG could significantly reduce IL-6 and TNFa production in LPS-activated mouse peritoneal macrophages, and NDX1 was the most effective among them. Without being bound by any theory, further studies using the human cells/tissues and the specific animal disease models may provide the necessary information on whether NDX1 is a viable strategy to target inflammation in a variety of pathologies, including but not restricted to osteoarthritis, rheumatoid arthritis, inflammatory bowel disease, and cancer.

1. A method of treating a subject having an inflammatory disease, such as rheumatoid arthritis (RA), osteoarthritis (OA), colitis, asthma, systemic sclerosis, Sjögren's syndrome, bronchiectasis, idiopathic pulmonary fibrosis, atherosclerotic plaques, ulcerative colitis, Crohn's disease, and the like, comprising administering a therapeutically effective amount of a compound of the formula 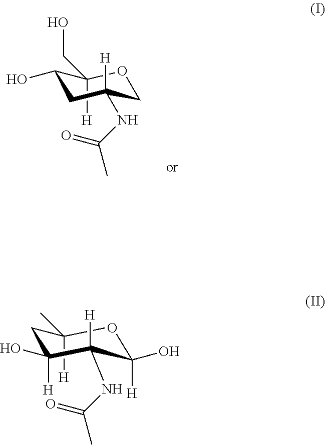
or a pharmaceutically acceptable salt thereof.

2. The method of claim 1, wherein the inflammatory disease is rheumatoid arthritis (RA).

3. The method of claim 1, wherein the inflammatory disease is osteoarthritis (OA).

4. The method of claim 1, wherein the therapeutically effective amount treats one or more symptoms of the inflammatory disease.

5. The method of claim 1, wherein the compound is administered intravenously, orally, subcutaneously, buccally, transdermally, or nasally.

6. The method of claim 1, wherein the compound is administered orally.

7. The method of claim 1, wherein the therapeutically effective amount of the inflammatory disease is in the range of about 1 mg to about 1000 mg.

8. The method of claim 1, wherein the therapeutically effective amount of the compound is administered once weekly, twice weekly, once a day (QD), twice a day (BID), or three times a day (TID).

9. The method of claim 1, wherein the therapeutically effective amount of the compound inhibits NO production in the patient.

10. The method of claim 1, wherein the therapeutically effective amount of the compound inhibits iNOS gene expression in the patient.

11. The method of claim 1, further comprising administering to the patient of one or more additional therapeutic agents.

12. The method of claim 11, wherein the one or more additional therapeutic agents is an a corticosteroid, a salicylate, an acetic acid derivative, an enolic acid (oxicam) derivative, a propionic acid derivative, an anthranilic acid derivative, or a Cox-2 inhibitor.

13. The method of claim 11, wherein the one or more additional therapeutic agents is selected from the group consisting of aspirin, diflunisal, salsalate, diclofenac, etodolac, indomethacin, ketorolac, nabumetone, sulindac, tolmetin, meloxicam, piroxicam, ibuprofen, naproxen, ketoprofen, fenoprofen, flurbiprofen, oxoprozin, mefenamic acid, celecoxib, cortisone, prednisone, and methylprednisone.

14-45. (canceled)

46. The method of claim 1, wherein the compound is of formula (I), or a pharmaceutically acceptable salt thereof.

47. The method of claim 1, wherein the compound is of formula (II), or a pharmaceutically acceptable salt thereof.

48. The method of claim 11, wherein the compound is of formula (I), or a pharmaceutically acceptable salt thereof.

49. The method of claim 11, wherein the compound is of formula (II), or a pharmaceutically acceptable salt thereof.

50. The method of claim 12, wherein the compound is of formula (I), or a pharmaceutically acceptable salt thereof.

51. The method of claim 12, wherein the compound is of formula (II), or a pharmaceutically acceptable salt thereof.


SEQUENCE LISTING (12 sequences)

SEQ ID NO: 1 - DNA, 20 bp, synthetic construct
  source 1..20: mol_type = other DNA
  misc_feature 1..20: note = forward primer for 18s rRNA
gtgaccagttcactcttggt

SEQ ID NO: 2 - DNA, 21 bp, synthetic construct
  source 1..21: mol_type = other DNA
  misc_feature 1..21: note = reverse primer for 18s rRNA
cattggaagtgaagcgtttcg

SEQ ID NO: 3 - DNA, 20 bp, synthetic construct
  source 1..20: mol_type = other DNA
  misc_feature 1..20: note = forward primer for Cox-2
tgcattctttgcccagcact

SEQ ID NO: 4 - DNA, 20 bp, synthetic construct
  source 1..20: mol_type = other DNA
  misc_feature 1..20: note = reverse primer for Cox-2
aaaggcgcagtttacgctgt

SEQ ID NO: 5 - DNA, 21 bp, synthetic construct
  source 1..21: mol_type = other DNA
  misc_feature 1..21: note = forward primer for IL-6
ggtacatcctcgacggcatct

SEQ ID NO: 6 - DNA, 21 bp, synthetic construct
  source 1..21: mol_type = other DNA
  misc_feature 1..21: note = reverse primer for IL-6
gtgcctctttgctgctttcac

SEQ ID NO: 7 - DNA, 21 bp, synthetic construct
  source 1..21: mol_type = other DNA
  misc_feature 1..21: note = forward primer for iNOS
gagggatgccttccgcagctg

SEQ ID NO: 8 - DNA, 23 bp, synthetic construct
  source 1..23: mol_type = other DNA
  misc_feature 1..23: note = reverse primer for iNOS
gaatcgaaccctgattccccgtc

SEQ ID NO: 9 - DNA, 25 bp, synthetic construct
  source 1..25: mol_type = other DNA
  misc_feature 1..25: note = forward primer for IL-1 beta
caaccaacaagtgatattctccatg

SEQ ID NO: 10 - DNA, 21 bp, synthetic construct
  source 1..21: mol_type = other DNA
  misc_feature 1..21: note = reverse primer for IL-1 beta
gatccacactctccagctgca

SEQ ID NO: 11 - DNA, 21 bp, synthetic construct
  source 1..21: mol_type = other DNA
  misc_feature 1..21: note = forward primer for IL-6
gagtccttcagagagatacag

SEQ ID NO: 12 - DNA, 18 bp, synthetic construct
  source 1..18: mol_type = other DNA
  misc_feature 1..18: note = reverse primer for IL-6
tggtcttggtccttagcc